(19) United States
(12) Patent Application Publication
(10) Pub. No.: US 2011/0081325 A1
(43) Pub. Date: Apr. 7, 2011

(54) NOVEL TREATMENT OF HEART DISEASES

(75) Inventor: Heribert Bohlen, Koln (DE)

(73) Assignee: Axiogenesis AG, Koln (DE)

(21) Appl. No.: 12/993,981

(22) PCT Filed: Jun. 2, 2009
(86) PCT No.: PCT/EP2009/003932

§ 371 Date: Nov. 22, 2010

(30) Foreign Application Priority Data
Jun. 2, 2008 (EP) 08010066.2

(51) Int. Cl. A61K 35/34; A61P 9/10; A61P 9/04; A61K 31/475; C12Q 1/02; C12N 5/00

(52) U.S. Cl. 424/93.7; 514/283; 435/29; 435/404

(57) ABSTRACT

Provided is a novel therapeutic approach for the treatment of heart failure and diseases associated therewith. In particular, vinca alkaloids are used for improving the viability of cardiomyocytes and preventing myocardial infarction.

### TECHNICAL FIELD

The present invention relates to the treatment of heart failure and diseases associated therewith.

### BACKGROUND OF THE INVENTION

Heart failure is the most prevalent cardiovascular disease, which results in a large number of hospital admissions and carries with it an extremely high mortality rate. It is estimated by the U.S. National Institutes of Health (NIH) that 4.8 million Americans have congestive heart failure, calling it a “new epidemic.” That number is projected to double by 2007. On average, 550,000 new cases are diagnosed with the often-fatal condition each year. By simple definition, heart failure is a chronic ailment where the heart fails to function normally due to impairment of the heart's pumping ability (left ventricular systolic dysfunction). However, what this diagnosis truly represents is a complex clinical syndrome that can develop from virtually any cardiac disorder of the pericardium, myocardium, endocardium, or great, vessels, but the majority of heart failure patients have symptoms due to the impairment of the left ventricular function. Damage to the left ventricle of the heart limits the ability of the ventricle to eject blood from the ventricle, resulting in an enlarged, weakened muscle, which can no longer squeeze effectively to pump the blood through the chamber.

Difficulty in breathing (dyspnea), asthma, ankle edema, and fatigue affect the patient's ability to perform their normal activities of daily living and are the hallmarks of the clinical manifestations of heart failure. Noticeably, the clinical picture of heart failure worsens to dominate the life of the patient: frequent physician office visits, numerous hospitalizations, multiple medications, with resultant side effects, and activity restrictions impacting on the quality of life for the patient with the consequential functional capacity impairment of the cardiopulmonary system. The prognosis of end-stage heart failure is dismal. Despite several decades of research, none of the mechanical assist devices and total artificial hearts has found widespread use due to several key technical limitations of these forms of therapy.

In view of the rapidly rising rates of new cases regardless of the differentiation of acute vs. chronic heart failure, treatment is primarily aimed at the underlying cause with concomitant therapy to improve cardiac performance. Therapeutic procedures include, multiple medications such as beta-blockers, diuretics, digoxin, antiarrhythmics, anticoagulants, implantable defibrillators and biventricular pacing, left ventricular assistance device therapy and cardiac transplantation (with limited availability due to the scarcity of donor hearts).

Since recently, stem cell therapy for heart disease is being used to improve the quality of life of patients suffering from coronary heart disease, congestive heart failure and cardiomyopathy and relieve symptoms such as severe angina pectoris (chest pain) and shortness of breath.

### SUMMARY OF THE INVENTION

The present invention provides a system and method for treating heart failure, including by the direct or indirect delivery of vinca alkaloids as the therapeutically active agent, or functional analogues or equivalents thereof, to the heart in a manner to induce revitalization and/or proliferation of myocardial cells with improvement or retention of cardiac function. The equivalents of vinca alkaloids include prodrugs, metabolites as well as compounds that share similar biological properties with vinca alkaloids such as tubulin dimer binding; see, e.g., Lobert et al., Biochemistry 35 (1996), 6806-6814.

The present invention provides an agent and related compositions for alleviating, including preventing, symptoms, see supra, and prolonging survival in patients with or at risk of heart failure. The agent and composition of this invention can be used for “treatment” in a preventive, curative, palliative, supportive, and/or restorative manner with respect to heart failure.

The present invention provides a system, including related compositions and methods, for the use of tubulin binding agents to improve myocardial performance. Preferred agents comprise vinca alkaloids, preferably selected from the group consisting of vinorelbin, vincristin, vinblastine and pharmaceutically acceptable salts and derivatives thereof.

The present invention also provides combination preparations and combined uses of tubulin binding agents such as preferably vinca alkaloids and other therapeutic means for improving cardiac function, for example transplants of, e.g., stem cells, cardiomyocytes, heart transplant or implantable cardioverter-defibrillator (ICD).

### BRIEF DESCRIPTIONS OF THE FIGURES

FIGS. 1-4: Effect of different vinca alkaloids (FIGS. 1-3) and doxorubicin (FIG. 4) on the viability and rate of survival of in vitro differentiated cardiomyocytes. EBs were generated and cardiac cells were differentiated as described in Example 1. On day 14, EBs were treated with vinca alkaloids and and doxorubicin as indicated in the figures. Photomicrographs were taken before treatment and 48 and 72 hours after treatment. Fluorescent areas representing cardiac cells were measured and calculated as described in Example 2 and 3. Cor.At refers to in vitro differentiated cardiomyocytes and MEF to Mouse Embryonic Fibroblasts.

### DETAILED DESCRIPTION OF THE INVENTION

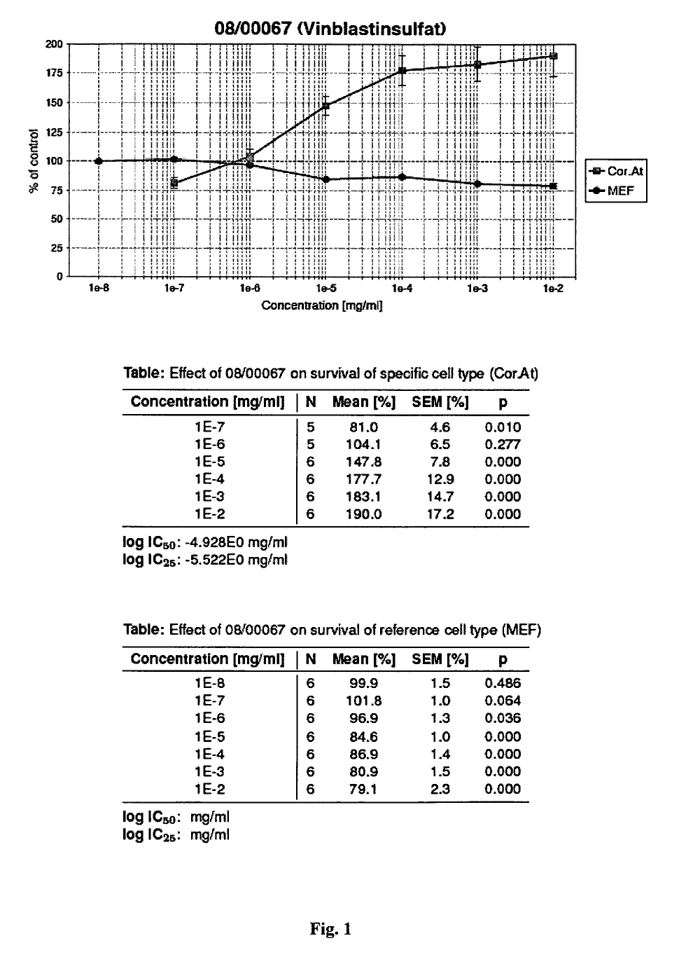

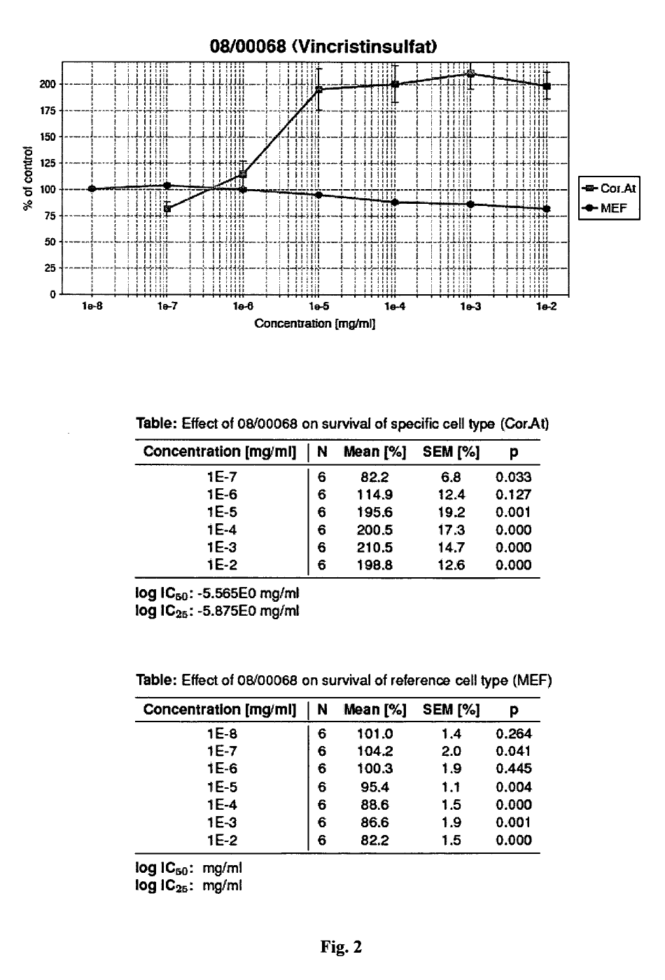

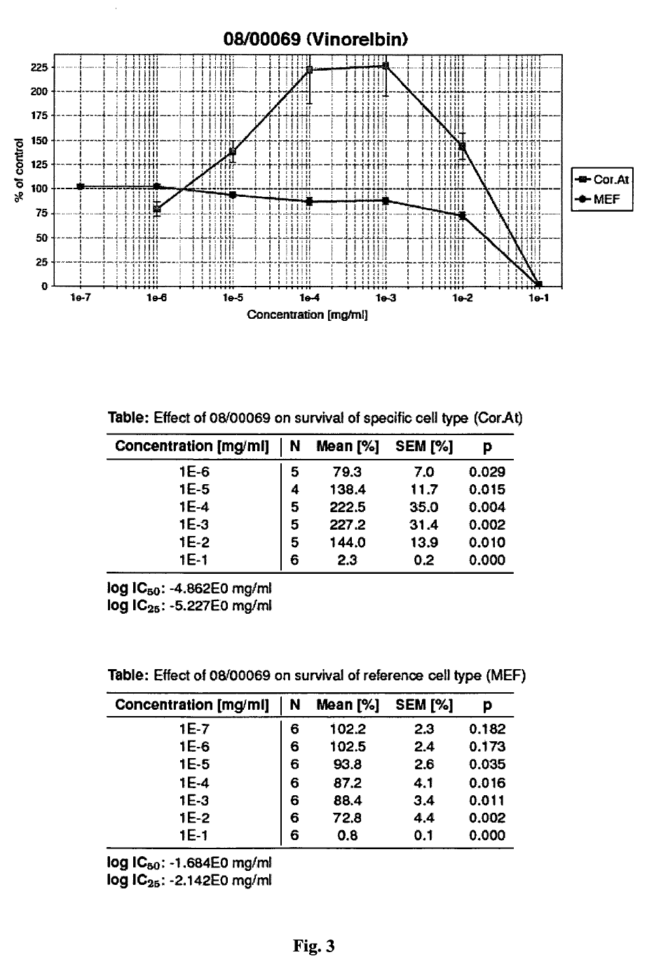

The present invention generally relates to vinca alkaloids and equivalent tubulin binding agents for the treatment of a heart disease such as heart failure, myocardial infarction or cardiomyopathy. The present invention is based on the surprising observation that vinca alkaloids, a type of mitotic inhibitor that blocks cell growth by interfering with microtubules and hitherto used to treat cancer, substantially improves the viability and survival rate of cardiomyocytes; see Example 2 and FIGS. 1 to 3.

The most prominent members of vinca alkaloids are vinorelbin, vincristin, and vinblastin, which are preferably used in accordance with the present invention. However, it is to be understood that the present invention extends to biologically active derivatives or functional analogues of vinca alkaloids, e.g., to analogues or derivatives of vinorelbin, vincristin, and vinblastin. The terms “analogue”, and “derivative” of vinca alkaloids mean a molecule which retains some or all of the biological function or activity as vinorelbin, vincristin, and vinblastin, in particular the capability of improving the viability of cardiomyocytes. This biological function can be tested in accordance with the appended Examples. Methods for the preparation of chemical derivatives and analogues are well known to those skilled in the art and are described in, for example, Beilstein, Handbook of Organic Chemistry, Springer edition New York Inc., 175 Fifth Avenue, New York, N.Y. 10010 U.S.A. and Organic Synthesis, Wiley, New York, USA. Derivatives of vinca alkaloids are known to the person skilled in the art and are described in the literature. For example, international applications WO2005/055939 and WO2008/033930 disclose derivatives of vinca alkaloids as well as processes of their preparation; international application WO2007/098091 describes vinca alkaloid N-oxide analogs.

The term “treatment” as used herein is intended to include either therapeutic treatment of heart failure, or preventive or prophylactic procedures performed before the occurrence of the disorder.

Further agents, which might be equivalent to vinca alkaloids and thus useful in accordance with the teaching of the present invention include but are not limited to colchicine, steganacin, podophyllotoxin, nocodazole, combretastatin, curacin A, dolastatin, 2-methoxyestradiol and dihydroxy-pentamethoxyflavanone as well as derivatives of any one thereof. Derivatives of those known tubulin-binding agents, for example in which a (poly)fluorobenzene, a fluoropyridine, or a fluoronitrobenzene moiety is incorporated or added to the core structure and strategies to therefor are disclosed in international application WO00/035865.

In accordance with present invention putative drugs for the treatment of a heart disease equivalent to vinca alkaloids but also the cardiotoxicity of a substance can be identified by a method comprising:

(a) contacting a test sample comprising in vitro differentiated cardiomyocytes with a test substance prior, during or after differentiation of the cardiomyocytes in the presence of a compound which induces a predefined diseased phenotype of the cardiomyocytes which substantially corresponds to a phenotype of a cell of a diseased cell, tissue or organ of a heart disease;
(b) determining a responsive change of the phenotype of the cells in said test sample, wherein a responsive change

(i) preventing or delaying the onset or the progression of the diseased phenotype is indicative for a useful drug; and

(ii) enhancing the onset or progression the diseased phenotype is indicative for the toxicity of the compound.

The method of the present invention can be performed in principle such as described in international application WO2005/108598, the disclosure content of which is incorporated herewith in its entirety, which generally discloses assay systems for determining the therapeutic or toxic effect of a putative drug based on assaying its activity in cells which have been differentiated in vitro from stem cells, and induced to display a phenotype that resembles a disease to be treated. Preferably, said disease is heart failure or a cardiomyopathy and the phenotype is survival of cardiomyocytes. As described in Example 3, a compound of known cardiotoxic potential such as doxorubicin may be used as the compound inducing the diseased phenotype.

Regarding the test substance any substance of interest may be used; see for example the test compounds and substances referred to in international application WO2005/108598. Nevertheless, advantageously substances derived from vinca alkaloids and other tubulin binding agents are used since they are expected to have great potential to exert similar effects like the vinca alkaloids exemplified in the Examples. Thus, also the vinca alkaloids referred to supra may be tested and improved, if necessary, as to their cardioactive properties.

The term “cardioactive” as used herein pertains to a drug or other substance affecting the function of the heart. For the purpose of the present invention a cardioactive agent is a substance which in an assay as described in Examples 2 and/or 3 exerts substantially the same or similar effects on cardiomyocytes as any one of the three vinca alkaloids exemplified herein. Thus, derivatives of vinca alkaloids and other tubulin binding agents which are equivalent to, for example, vincristin are supposed to be cardioactive.

In this context, the present invention also relates to a kit and composition containing a tubulin binding agent as defined above and stem cells or in vitro differentiated cardiomyocytes; optionally further comprising differentiation promoting compounds, culture medium, and/or test substances. Appropriate supplementary ingredients of the kit and composition are disclosed in, e.g., international application WO2005/108598 and WO2005/005621. The kit of the present invention is permeably used for the identification and investigation of putative cardioactive drugs and the toxicity of a given compound; see also supra.

Substances are metabolized after their in vivo administration in order to be eliminated either by excretion or by metabolism to one or more active or inactive metabolites (Meyer, J. Pharmacokinet. Biopharm. 24 (1996), 449-459). Thus, rather than using the actual vinca alkaloid or drug identified and obtained in accordance with the methods of the present invention a corresponding formulation as a pro-drug can be used which is converted into its active form in the patient by his/her metabolism. Precautionary measures that may be taken for the application of pro-drugs and drugs are described in the literature; see, for review, Ozama, J. Toxicol. Sci. 21 (1996), 323-329.

For therapeutic application, a therapeutically effective amount of the agent will usually be formulated as a pharmaceutical composition with a pharmaceutical acceptable carrier. The term “therapeutically effective amount” as used herein means that amount necessary at least partly to attain the desired effect, e.g., regeneration and/or preservation of heart tissue. Such amounts will depend on the particular injury being treated, the severity of the injury, and the characteristics of the individual subject, including age, physical condition, size, weight and other concurrent treatment, and will be at the discretion of the attending physician or veterinarian. Preferably the agent is administered by localized administration. Such administration may be achieved directly at the site, for example by one or more intrapericardial injections or implants, or with a delivery system. Alternatively, other modes of administration, such as systemic injections, may be used, provided that they increase the amount of the agent within heart tissue to attain the desired effect.

Methods and pharmaceutical carriers for the preparation of pharmaceutical compositions, including compositions for intrapericardial and intravenous administration, are well known in the art, as set out in textbooks such as Remington: The Science and Practice of Pharmacy (2000) by the University of Sciences in Philadelphia, ISBN 0-683-306472. Suitable pharmaceutically acceptable carriers and/or diluents include conventional solvents, saline solutions, dispersion media, fillers, aqueous solutions, antibacterial and antifungal agents and absorption-promoting agents. Except insofar as any conventional medium or agent is incompatible with the active ingredient, its use in the pharmaceutical compositions of the present invention is contemplated. Supplementary active ingredients such as cardioactive compounds which have the ability to promote healing or to inhibit inflammation may also be incorporated into the compositions. For example, the pharmaceutical composition may additionally include one or more other cytokines, including but not limited to insulin, epidermal growth factor, fibroblast growth factor, insulin-like growth factor, betacellulin, transforming growth factor alpha or transforming growth factor beta. In one embodiment, the pharmaceutical composition is devoid of therapeutically effective amounts of any other chemotherapeutic compound, in particular those used in cancer therapy.

In one particular preferred embodiment of the present invention the agent is used in stem cell therapy and cell or organ transplantation. As evident from Example 2, vinca alkaloids substantially improve the survival of in vitro differentiated cardiomyocytes. The viability and survival of cardiomyocytes is also a critical feature for the successful transplantation of fetal or stem cell derived cardiomyocytes as well as of heart transplants. Furthermore, the viability of cardiomyocytes is important in heart surgery, for example stent and cardiac pacemaker implantation which could stress or injure the cardiac tissue. Accordingly, the present invention also relates to combination preparations and the combined use of vinca alkaloids and equivalent tubulin binding agents in combination with a transplant selected from stem cells, cardiomyocytes, heart transplant, stent, cardiac pacemaker or implantable cardioverter-defibrillator (ICD).

In this context, the present invention also pertains to cell, tissue and organ culture media containing a vinca alkaloid or equivalent agent in an amount sufficient to maintain or improve viability and survival, respectively, of the cell, tissue and organ compared to a culture medium without said vinca alkaloid or agent. Preferably, the cell, tissue and organ is a stem cell or cardiomyocyte, cardiac tissue and heart, respectively.

The pharmaceutical composition may be present in a form suitable for oral, rectal, transdermal, dermal, ophthalmological, nasal, pulmonary or parenteral application. Preferably, the pharmaceutical preparation is suited for oral administration. It may then be present in the form of tablets, coated tablets, capsules, granulate, solutions for drinking, liposomes, nano-particles, nano-capsules, micro-capsules, micro-tablets, pellets or powders and in the form of granulate filled in capsules or sachets, micro-tablets filled in capsules or sachets, pellets filled in capsules or sachets, nano-particles filled in capsules or sachets or powder filled in capsules or sachets. Preferably, the drug is present in the form of nano-particles, pellets or micro-tablets, which may optionally be filled in sachets or capsules.

Preferably, all solid oral dosage forms may be provided with an enteric coating. It may e.g. be applied onto the tablets, micro-tablets, pellets, etc., but may also be applied onto the capsules that contain them.

In the case of a parenteral administration via an injection (iv, im, sc, ip) the preparation is present in a form suitable for this. All customary liquid carriers suitable for the injection can be used; see also Remington (2000), supra. For example, a stable, injectable pharmaceutical composition of vinca alkaloid salts, see also Example 2 and FIGS. 1 to 3, can be in the form of an aqueous solution comprising per 1 ml of solution: from about 0.2 to about 2 mg of one or more pharmaceutically acceptable vinca alkaloid salts; from about 0.1 to about 1.0 mg of a pharmaceutically acceptable ethylenediamine-tetraacetic acid (EDTA) salt; acetate buffer in an amount necessary to maintain said aqueous solution at a pH of from about 3.0 to about 5.5; and from about 1.5 to about 2.5 mg of a preservative selected from methyl paraben, propyl paraben and mixtures thereof. Preferably, the vinca alkaloid or derivatives are present in the pharmaceutical composition in an amount per dosage unit which corresponds and/or is equivalent to an amount of 1 to 500 mg, preferably 10 to 300 mg, and mostly preferred 10 to 200 mg of the agent. In particular, the pharmaceutical composition is preferably designed to be administered in a dose equivalent to about 0.1 to 10 mg of vincristine per square meter (m²) intravenously, preferably 0.5 to 5 mg, more preferably about 1.0 to 2.5 and most preferably about 1.4 mg of vincristine per square meter (m²) intravenously (maximal dose, 2.0 mg). This dose may be administered to result in a predicted dose-intensity of 0.1 to 1.0, preferably to 0.4 to 0.7 mg/m²/week. Dose reductions can be made as previously described if there is hematologic toxicity; see, e.g., McKelvey et al., Cancer 38 (1976), 1484-1493; however, the first course may be given in full. If there is mild neurotoxicity (i.e., paresthesias or decreased tendon reflexes), the dose of the vinca alkaloid may be reduced to 50 percent. In this context, it will be noted that in accordance with the experiments performed within the scope of the present invention vinca alkaloids exert their are cardioactive effect in an amount of 1E-6 and 1E-5 mg/ml already and reach their maximum activity at a concentration at which the reference cell type still remains substantially unaffected. Thus, the physician will have a rather broad concentration range at which the vinca alkaloids and equivalent tubulin binding agents can be administered and used for treating a heart disease while avoiding or at least minimizing any side effects of the agent being due to its known effect on tubulin structures.

The administrations contemplated by the present invention include administration of any formulations suitable for delivery of the agent, such as aqueous isotonic solutions, suspensions, gels, and polymers impregnated with the agent, or for topical administration of the agent, such as aqueous creams, ointments, gels, lotions, sprays, microspheres, liposomes, wound dressings, and synthetic polymer dressings or sutures impregnated with the agent, and the like.

The agent of this invention can be delivered in any suitable manner and using any suitable means, including in vehicles that comprise liquids, solids, semisolids, matricies, powders, and/or particles, and which in turn can be bioabsorbable, biodegradable or stable. Emerging technologies can also be relied upon, including medicated powders pumped into the tissue at supersonic speeds, implanted biochips, and nanomolecular transportation systems. In turn, the vehicle can be delivered to the desired site with or without the agent, and using any suitable means, including by open or minimally invasive access, using catheters, membranes, lasers, or other medical-surgical instruments. Suitable delivery routes include, but are not restricted to, intramuscular, subcutaneous, percutaneous, oral, transdermal, intranasal, ocular, intrapericardial, direct myocardial injection, percutaneously through the left ventricular cavity or surgically through the epicardium, as well as infusion into the pericardial sac using implantable and/or external pumps.

The pharmaceutical composition and therapeutic methods of the present invention are complementary to existing forms of therapy for heart failure, including mechanical devices, electrophysiological forms of therapy, and pharmacological agents. Since the vinca alkaloids can be used to revitalize and repair the myocardium, they can be used with most, if not all, patients with overt failure, and can be considered for asymptomatic patients with left cardiac dysfunction as well. The system can be coupled with ease-of-use through minimally invasive techniques. Since the therapy results in cardiomyocytes being revitalized, it is expected to become the standard of care.

Furthermore, in view of the results obtained by the experiments performed in accordance with the present invention it also relates to the above described tubulin binding agents for neutralizing/antagonizing the side-effects of an anti-tumor agent, preferably selected from DNA intercalating compounds such as doxorubicin on cardiomyocytes as well as on cardiac tissue in general; see also Example 3. Hence, the present invention generally relates to the use of tubulin binding agents, in particular vinca alkaloids and derivatives thereof for the preparation of a composition for increasing the viability of cardiomyocytes and to methods of increasing the viability of cardiomyocytes, cardiac tissue or heart transplant in subject suffering from a heart disease, said method comprising administering to a subject in need thereof a therapeutically effective amount of a vinca alkaloid or equivalent tubulin binding agent.

These and other embodiments are disclosed and encompassed by the description and examples of the present invention. Further literature concerning any one of the materials, methods, uses and compounds to be employed in accordance with the present invention may be retrieved from public libraries and databases, using for example electronic devices. For example the public database “Medline” may be utilized, which is hosted by the National Center for Biotechnology Information and/or the National Library of Medicine at the National Institutes of Health. Further databases and web addresses, such as those of the European Bioinformatics Institute (EBI), which is part of the European Molecular Biology Laboratory (EMBL) are known to the person skilled in the art and can also be obtained using internet search engines. An overview of patent information in biotechnology and a survey of relevant sources of patent information useful for retrospective searching and for current awareness is given in Berks, TIBTECH 12 (1994), 352-364.

Several documents are cited throughout the text of this specification. The contents of all cited references (including literature references, issued patents, published patent applications as cited throughout this application and manufacturer's specifications, instructions, etc) are hereby expressly incorporated by reference; however, there is no admission that any document cited is indeed prior art as to the present invention.

The above disclosure generally describes the present invention. A more complete understanding can be obtained by reference to the following specific examples which are provided herein for purposes of illustration only and are not intended to limit the scope of the invention.

### EXAMPLES

The examples which follow further illustrate the invention, but should not be construed to limit the scope of the invention in any way. Detailed descriptions of conventional methods, such as those employed herein can be found in the cited literature; see also “The Merck Manual of Diagnosis and Therapy” Seventeenth Ed. ed by Beers and Berkow (Merck & Co., Inc. 2003).

The practice of the present invention will employ, unless otherwise indicated, conventional techniques of cell biology, cell culture, molecular biology, transgenic biology, microbiology, recombinant DNA, and immunology, which are within the skill of the art.

For further elaboration of general techniques concerning stem cell technology, the practitioner can refer to standard textbooks and reviews, for example Teratocarcinomas and embryonic stem cells: A practical approach (E. J. Robertson, ed., IRL Press Ltd. 1987); Guide to Techniques in Mouse Development (P. M. Wasserman et al., eds., Academic Press 1993); Embryonic Stem Cell Differentiation in Vitro (Wiles, Meth. Enzymol. 225 (1993), 900,); Properties and uses of Embryonic Stem Cells: Prospects for Application to Human Biology and Gene Therapy (Rathjen et al., Reprod. Fertil. Dev. 10 (1998), 31,). Differentiation of stem cells is reviewed in Robertson, Meth. Cell Biol. 75 (1997), 173; and Pedersen, Reprod. Fertil. Dev. 10 (1998), 31. Besides the sources for stem cells described already above further references are provided; see Evans and Kaufman, Nature 292 (1981), 154-156; Handyside et al., Roux's Arch. Dev. Biol., 196 (1987), 185-190; Flechon et al., J. Reprod. Fertil. Abstract Series 6 (1990), 25; Doetschman et al., Dev. Biol. 127 (1988), 224-227; Evans et al., Theriogenology 33 (1990), 125-128; Notarianni et al., J. Reprod. Fertil. Suppl., 43 (1991), 255-260; Giles et al., Biol. Reprod. 44 (Suppl. 1) (1991), 57; Strelchenko et al., Theriogenology 35 (1991), 274; Sukoyan et al., Mol. Reprod. Dev. 93 (1992), 418-431; Iannaccone et al., Dev. Biol. 163 (1994), 288-292.

Methods in molecular genetics and genetic engineering are described generally in the current editions of Molecular Cloning: A Laboratory Manual, (Sambrook et al., (1989) Molecular Cloning: A Laboratory Manual, 2nd ed., Cold Spring Harbor Laboratory Press); DNA Cloning, Volumes I and II (D. N. Glover ed., 1985); Oligonucleotide Synthesis (M. J. Gait ed., 1984); Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Gene Transfer Vectors for Mammalian Cells (Miller & Calos, eds.); Current Protocols in Molecular Biology and Short Protocols in Molecular Biology, 3rd Edition (F. M. Ausubel et al., eds.); and Recombinant DNA Methodology (R. Wu ed., Academic Press). Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.); Immobilized Cells And Enzymes (IRL Press, 1986); B. Perbal, A Practical Guide To Molecular Cloning (1984); the treatise, Methods In Enzymology (Academic Press, Inc., New York); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986). Reagents, cloning vectors, and kits for genetic manipulation referred to in this disclosure are available from commercial vendors such as BioRad, Stratagene, Invitrogen, and Clontech. General techniques in cell culture and media collection are outlined in Large Scale Mammalian Cell Culture (Hu et al., Curr. Opin. Biotechnol. 8 (1997), 148); Serum-free Media (Kitano, Biotechnology 17 (1991), 73); Large Scale Mammalian Cell Culture (Curr. Opin. Biotechnol. 2 (1991), 375); and Suspension Culture of Mammalian Cells (Birch et al., Bioprocess Technol. 19 (1990), 251). Other observations about the media and their impact on the culture environment have been made by Marshall McLuhan and Fred Allen.

### Example 1

### Identification of Cardioactive Compounds

The generation of “embryoid bodies” (EBs) from high density cell suspensions and differentiation of cardiac cells was essentially performed as described in Example 1 of international application WO2005/005621, the disclosure content of which is hereby incorporated by reference in its entirety. Briefly, mouse embryonic stem cells (ES cells, clone D3, ATCC CRL 1934) were stably transfected with the paMHC-GFP vector containing the gene of the green fluorescent protein under control of the cardiac α-myosin heavy chain (α-MHC) promotor. To obtain this vector, a 5.5 kb fragment containing the promoter region of the mouse α-myosin heavy chain gene (Genbank 471441) was introduced into the polylinker of the pEGFP-1 vector (Clontech Laboratories).

ES cells were cultured on 10 cm petri dishes (Falcon, Becton Dickinson) at a density of 1.4×10⁶ in DMEM (Gibco, Invitrogen) supplemented with 15% FCS (Gibco, invitrogen, batch controlled) and 1×10³ U/ml LIF (Chemicon) on a layer of feeder cells (inactivated mouse embryonic fibroblasts, prepared according standard protocols; see also description of international application WO2005/005621). Cells were incubated at 37° C., 7% CO₂ and 95% humidity. Cells were split every second day by trypsinizing them to single cell suspension and seeding 1.4×10⁶ on a fresh 10 cm dish coated with feeder cells.

ES cells from one or more petri dishes were trypsinized to obtain a single cell suspension and collected by centrifugation (800 g for 5 min). Cells were resuspended to a density of 2×10⁶ cells/ml in Iscove's Modified Dulbecco's Medium (IMDM, Invitrogen) supplemented with 20% (v/v) fetal bovine serum (FBS, Invitrogen, batch controlled).

To generate EBs, ES cells were cultured in suspension at a density of 2×10⁶ cells/ml in a 6 cm petri dish (Greiner, Darmstadt, Germany) in 4 ml IMDM with 20% FCS (Invitrogen, Karlsruhe, Germany) at 37° C., 5% CO₂, 95% humidity on a rocking table (GFL 3006, GFL, Braunschweig, Germany) at 50 rpm for 6 hours. After 6 hours the suspension was diluted 1:10 with IMDM with 20% FCS and incubated for additional 12-16, preferably to a total of 18 hours in T25 cell culture flasks (Falcon, Becton Dickinson, Heidelberg, Germany) on the rocking table at 37° C., 5% CO₂, 95% humidity. On the next day, EB suspension was transferred to a COPAS select particle sorter (Union Biometrica, Geel, Belgium) and single EBs were sorted into the wells of 96-well U-shaped microtiter plates (Greiner) according to the manufacturer's instructions. EBs were cultured in 200 μl IMDM 20% FCS per well and incubated at 37° C., 5% CO₂, 95% humidity. On day 5 and 10, the medium was replaced by fresh medium. On day 14, fluorescent areas representing cardiac cells were detected by fluorescence microscopy using a Zeiss Axiovert 200M with a 10x Achroplan objective, a HQ-filterset for GFP (AF Analysentechnik, Tubingen, Germany) and a Sensicam 12 bit cooled imaging system (PCO Imaging, Kelheim, Germany). In addition, or alternatively, the generation of EBs is performed from low density cell suspensions with subsequent differentiation of cardiac cells as described in Example 2 and protocol 2 of international application WO2005/005621.

Assaying test compounds was essentially performed as described in Examples 3 and 4 of international application WO2005/005621. Control compounds can be chosen from a list of compounds recommended for a validation study on in vitro embryotoxicity tests by the European Center for the Validation of Alternative Methods (ECVAM) (see Brown, N A 2002; ATLA 30, 177-198). Compounds can be judged as cardioactive or embryotoxic, if a significant alteration in differentiation and/or survival of EBs and cardiomyocytes, respectively, is seen (Student's t-test).

### Example 2

### Vinca Alkaloids Vinblastin, Vincristin and Vinorelbin Increase Survival of in Vitro Differentiated Cardiomyocytes

EBs were generated as described in Example 1. On day 5, 5 EBs were transferred into each well of a 24-well tissue culture plate (Falcon, Becton Dickinson) into 2 m1 of IMDM 20% FCS and incubated at 37° C., 5% CO₂, 95% humidity. Half of the medium was replaced by fresh medium at day 10. At day 14, EBs were evaluated for cardiac differentiation by fluorescence microscopy, and fluorescence microphotographs of EBs with fluorescent areas were taken using a Zeiss Axiovert 200M with a 10× Achroplan objective, a HQ-filterset for GFP (AF Analysentechnik) and a Sensicam 12 bit cooled imaging system (PCO Imaging).

Test compounds were added at different concentrations as indicated in FIGS. 1 to 3 (solvent: DMSO, final concentration of DMSO: 0.1%), 0.1% DMSO was used as a negative control. EBs were incubated at 37° C., 5% CO₂, 95% humidity additional 3 days. After 48 hours and 72 hours of incubation with the compounds, fluorescence photomicrographs were taken, and the fluorescent areas were calculated using AnalySIS software (Soft Imaging Systems, Munster, Germany). Values obtained after treatment with the test compounds were compared with the values obtained before the treatment.

FIGS. 1 to 3 show the effects of 3 different vinca alkaloids, namely vinblastin, vincristin and vinorelbin to increase survival of in vitro differentiated cardiomyocytes on ES cell-derived cardiomyocytes after 72 hours. Moreover, as can be seen from the figures the cardioactive effect of the vinca alkaloids is achieved at a concentration at which the reference cell remains substantially unaffected in kind. Thus, there is no need of local administration since side effects of the vinca alkaloids do not seem to appear at a concentration which is therapeutically effective for the treatment of heart diseases.

### Example 3

### Vinblastin, Vincristin and Vinorelbin Neutralize Cardiotoxic Compounds

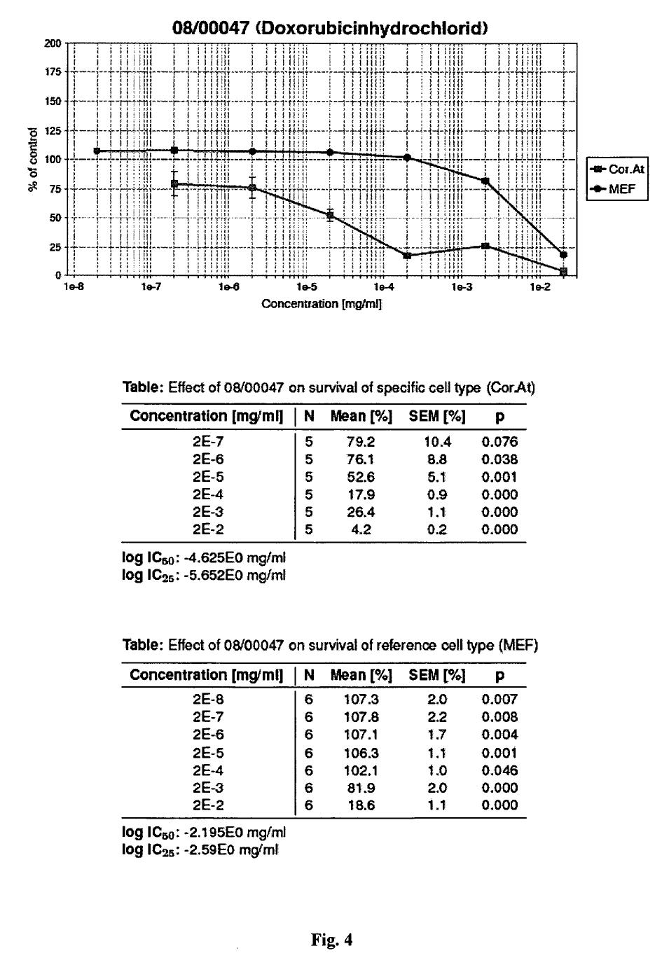

First, the cardiotoxic effect of doxorubicin was indentified in accordance with Example 4 of international application WO2005/005621; see also FIG. 4. Thereafter, Example 2, supra, is modified in that doxorubin (doxorubicinhydrochlorid) together with vinblastin, vincristin or vinorelbin is added at different concentrations and reversed dilution series to the cell culture.

After 48 hours and 72 hours of incubation with the compounds, fluorescence photomicrographs are taken, and the fluorescent areas are calculated using AnalySIS software (Soft Imaging Systems, Munster, Germany). Values obtained after treatment with the test compounds are compared with the values obtained before the treatment and with the treatment with doxorubicin alone.

Surprisingly, the cardiotoxic effect of doxorubicin can be neutralized by any one of the three vinca alkaloids and may be even superseded by their cardioactive effect. Thus, vinca alkaloids may also prove useful in the prevention and amelioration of side effects during, for example cancer chemotherapy in which anti-cancer drugs such as doxorubicin and other anthracycline antibiotics, e.g., daunorubicin (Cerubidine, DaunoXome) and idarubicin (Idamycin) can cause severe heart damage.

1. A method for treating heart disease, comprising administering to a subject an agent selected from a vinca alkaloid or derivative or analog thereof, or a pharmaceutically acceptable salt thereof.

2. The method of claim 1, wherein the heart disease is heart failure, myocardial infarction or cardiomyopathy.

3. The method of claim 1, wherein the agent is a vinca alkaloid selected from the group consisting of vinorelbin, vincristin, vinblastin or any derivative thereof.

4. The method of claim 1, wherein the agent is formulated as a pharmaceutical composition with a pharmaceutical acceptable carrier.

5. The method of claim 4, wherein the pharmaceutical composition, further comprises a cardioactive compound.

6. The method of claim 4, wherein the pharmaceutical composition is devoid of any other type of chemotherapeutic compound.

7. A method for identifying and obtaining a drug for the treatment of a heart disease and for determining a cardiotoxicity of a substance, respectively, comprising:
(a) contacting a test sample comprising hi vitro differentiated cardiomyocytes with a test substance prior, during or after differentiation of the cardiomyocytes in the presence of a compound which induces a predefined diseased phenotype of the cardiomyocytes which substantially corresponds to a phenotype of a cell of a diseased cell, tissue or organ of a heart disease;
(b) determining a responsive change of the phenotype of the cells in said test sample, wherein a responsive change (i) preventing or delaying the onset or the progression of the diseased phenotype is indicative for a useful drug; and
(ii) enhancing the onset or progression the diseased phenotype is indicative for the toxicity of the compound.

8. The method of claim 7, wherein said disease is heart failure or a cardiomyopathy and said phenotype is survival of cardiomyocytes.

9. The method of claim 7, wherein said compound is doxorubicin.

10. The method of claim 7, wherein the test substance is a tubulin binding agent.

11. A drug obtainable by the method of claim 7 for neutralizing/antagonizing the side effects of an anti-tumor agent on cardiomyocytes.

12. A composition comprising a vinca alkaloid or equivalent tubulin binding agent in combination with an implant selected from stem cells, cardiomyocytes, heart transplant, stent, cardiac pacemaker or implantable cardioverter-defibrillator (ICD).

13. A stem cell culture medium comprising a vinca alkaloid or equivalent tubulin binding agent.

14. A kit or composition containing a vinca alkaloid or equivalent tubulin binding agent and stem cells or in vitro differentiated cardiomyocyte.

15. A method of increasing the viability of cardiomyocytes, cardiac tissue or heart transplant in subject suffering from a heart disease, said method comprising administering to a subject in need thereof a therapeutically effective amount of a vinca alkaloid or equivalent tubulin binding agent.

16. The kit of claim 14, further comprising differentiation promoting compounds, culture medium, and/or test substances.
